(19) United States
(12) Patent Application Publication
(10) Pub. No.: US 2024/0245638 A1
(43) Pub. Date: Jul. 25, 2024

(54) Method of Treating Parkinson's Disease

(71) Applicant: Clexio Biosciences Ltd., Jerusalem (IL)

(72) Inventors: Dorit MIMROD, Jerusalem (IL); Elijahu BERKOVICH, Jerusalem (IL); Dvora IZGELOV, Jerusalem (IL)

(73) Assignee: Clexio Biosciences Ltd., Jerusalem (IL)

(21) Appl. No.: 18/683,935

(22) PCT Filed: Aug. 19, 2022
(86) PCT No.: PCT/IB2022/057814

§ 371 Date: Feb. 15, 2024

Related U.S. Application Data
(60) Provisional application No. 63/234,801, filed on Aug. 19, 2021.
(60) Provisional application No. 63/315,183, filed on Mar. 1, 2022.

(51) Int. Cl. A61K 31/198; A61K 9/48

(52) U.S. Cl. CPC A61K 31/198; A61K 9/4825

(57) ABSTRACT

The disclosure is directed to methods of treating Parkinson's disease comprising administration of a capsule comprising an anti-parkinsonian medication and where the capsule's length is at least about 29 mm and the capsule's width at least about 9.5 mm.

### CROSS-REFERENCE TO RELATED APPLICATIONS

The present application claims the benefit of priority to U.S. Provisional Application Nos. 63/234,801, filed Aug. 19, 2021, and 63/315,183, filed Mar. 1, 2022, the entire contents of both of which are incorporated herein by reference.

### BACKGROUND

Parkinson's disease (PD) is a common progressive neurodegenerative disorder of the Central Nervous System (CNS) that mainly affects a sufferer's motor function. Symptoms develop slowly over time and increase in severity as the disease becomes more advanced. In early stage PD, patients typically present with tremors and other motor symptoms. Changes in posture and facial expressions are also seen. As the disease progresses, the symptoms become progressively worse and patients suffer from rigidity, walking problems, loss of balance, a general slowing of movements and episodes of akinesia. Eventually the disease advances to debilitating levels where the patient might no longer be able to stand or walk. PD can also affect non-motor functions and is associated with dementia, sensory problems, autonomic dysfunction, and sleep and emotional problems.

A number of scales exist which are used for identifying the severity of a PD sufferer's condition and which allow for monitoring of its progression over time. These include standard scales, such as the Unified Parkinson's Disease Rating Scale (UPDRS), the Movement Disorder Society-sponsored revision of the Unified Parkinson's Disease Rating Scale (MDS-UPDRS) and the Hoehn and Yahr scales, each of which are commonly used by practitioners in the field.

One of the more common and distressing symptoms of PD is oropharyngeal dysphagia, where the sufferer has alterations in their swallowing processes resulting in difficulty swallowing food and medication. The impact of dysphagia on PD sufferers is greater, however, than just affecting their swallowing habits and is associated with serious clinical complications such as loss of quality of life (QoL), insufficient medication intake, malnutrition, dehydration and it can even lead to aspiration pneumonia, the leading cause of death in patients with PD (Suttrup 2016).

The prevalence of dysphagia in the PD population has been identified by both subjective and objective analyses. The subjective analyses typically result from review of patient records or patient questionnaires and suffer, on occasion, from a lack of consistency in identifying the patient populations (Takizawa 2016). Objective analyses report on the prevalence after invasive investigations of PD sufferers using tests such as Fiberoptic Endoscopic Evaluation of Swallowing (FEES) or VideoFluoroscopic Swallowing Study (VFSS). Rather surprisingly, the objective analyses indicate that the prevalence of dysphagia in the PD population is much higher than the sufferers themselves realize subjectively (35%), with a prevalence found in of over 80% of sufferers (Kalf 2012).

A variety of studies have also attempted to identify whether a relationship exists between the PD sufferer's presentation of dysphagia and the overall degree of progression of their condition. Here too, objective testing of mixed stage PD populations has tended to identify that dysphagia severity is strongly correlated with the Hoehn and Yahr scale (Mohamed 2018, Buhmann 2019) and presents with an Hoehn and Yahr score of 2 or more (Clarke 1998, Ding 2018, Shilimkar 2020).

In addition to the clinical concerns that dysphagia creates, PD sufferers also have a considerable pill burden due to the low bioavailability and short half-life of their most commonly used oral medication, levodopa. Levodopa, and other anti-parkinsonian medications, are widely available commercially in a variety of solid oral dosage forms as both tablets and capsules. These forms typically range from small round tablets of 6 or 7 mm diameter to capsules, or oval tablets, of 17 to 18 mm lengths. In subjective analysis of a large general population of average age over 60 years old, solid oral dosage form size was considered to provide the most difficulty in swallowing (75%), proportionately capsules, whether hard-shell or softgel, were considered to cause more difficulty than any other type of dosage form, and only capsule lengths below 18 mm were thought to not cause difficulty swallowing (Schiele 2013). In a recent objective study of a mixed population of PD sufferers, a small to medium size effect favoring 18 mm hard-shell capsules over 17 mm oval tablets was found (Buhmann 2019).

Surprisingly, and in stark contrast to the established thinking in the art, it has now been found both objectively and subjectively, that PD sufferers swallowing very large capsules provides for better and safer swallowing thereby allowing for improved and easier medication administration for patients.

### SUMMARY OF THE INVENTION

Provided herein are methods of treating Parkinson's disease comprising orally administering to a subject diagnosed with a Hoehn and Yahr rating of at least 2, a capsule comprising an anti-parkinsonian medication and where the capsule's length is at least about 29 mm and the capsule's width at least about 9.5 mm.

Also provided are methods of administering an anti-parkinsonian medication to a subject diagnosed with a Hoehn and Yahr rating of at least 2, a capsule comprising the anti-parkinsonian medication where the capsule's length is at least about 29 mm and the capsule's width at least about 9.5 mm.

Also provided are methods of improving the swallowability of an anti-parkinsonian medication in a subject diagnosed with a Hoehn and Yahr rating of at least 2, comprising administering the medication within a capsule with a length of at least about 29 mm and a width of at least about 9.5 mm.

### DETAILED DESCRIPTION

The present inventions may be understood more readily by reference to the following detailed description taken in connection with the accompanying examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.” The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc. . . . up to 5 hours.

The present disclosure provides, inter alia, methods of treating Parkinson's disease comprising orally administering to a subject diagnosed with a Hoehn and Yahr rating of at least 2, a capsule comprising an anti-parkinsonian medication and where the capsule's length is at least about 29 mm and the capsule's width at least about 9.5 mm.

The present disclosure also provides, inter alia, methods of administering an anti-parkinsonian medication to a human subject diagnosed with a Hoehn and Yahr rating of at least 2, comprising orally administering to the subject a capsule comprising an outer shell and a composition comprising an the anti-parkinsonian medication, where the capsule's length is at least about 29 mm and the capsule's width at least about 9.5 mm.

As used herein, the term “treating Parkinson's disease” shall refer to the management of the symptoms of Parkinson's disease and/or to the slowing down of the progression of the condition.

As used herein, the term “Hoehn and Yahr rating” refers to the scoring or rating according to either the original Hoehn and Yahr scale (Hoehn 1967), or the modified version which introduced Stages 0, 1.5 and 2.5 (Goetz 2004). For example, a Hoehn and Yahr rating, or score, of 2 relates to a patient with symptoms on both sides but no impairment of balance whereas a rating, or score, of 2.5, relates to a patient with mild symptoms on both sides, with recovery when the ‘pull’ test is given.

In one embodiment of the invention, the subject has been diagnosed with suffering from Parkinson's disease and of having a Hoehn and Yahr rating of at least 2. In another embodiment of the invention, the subject has been diagnosed with suffering from Parkinson's disease and of having a Hoehn and Yahr rating of at least 2.5, at least 3 or at least 4.

In one embodiment of the invention, the subject has been diagnosed with suffering from Parkinson's disease and of having a Hoehn and Yahr rating of 2. In another embodiment of the invention, the subject has been diagnosed with suffering from Parkinson's disease and of having a Hoehn and Yahr rating of 2.5. In another embodiment of the invention, the subject has been diagnosed with suffering from Parkinson's disease and of having a Hoehn and Yahr rating of 3. In another embodiment of the invention, the subject has been diagnosed with suffering from Parkinson's disease and of having a Hoehn and Yahr rating of 4. In another embodiment of the invention, the subject has been diagnosed with suffering from Parkinson's disease and of having a Hoehn and Yahr rating of 5.

As used herein, the term “diagnosed” or “diagnosis of” refers to the most recent clinical evaluation of the subject by a suitably qualified healthcare professional.

As used herein, the term “capsule” refers to a solid oral dosage form comprising a composition of one or more active pharmaceutical ingredients and possibly additional excipients, and an outer shell. The capsule's outer shell will typically be physiologically inert and, once orally ingested by the subject, easily and quickly digested, releasing its contents into the gastrointestinal tract.

As used herein, the term “capsule length” or “capsule's length” refers to the measurement of the greatest of the three external dimensions of the capsule.

In one embodiment of the invention, the capsule's length is at least about 29 mm. In another embodiment of the invention, the capsule's length is at least about 29 mm, at least about 29.5 mm, at least about 30 mm, at least about 30.5 mm, at least about 31 mm or at least about 31.5 mm.

In one embodiment of the invention, the capsule's length is about 29 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm.

In one embodiment of the invention, the capsule's length is at least about 29 mm and less than about 32 mm. In another embodiment of the invention, the capsule's length is at least about 29 mm and less than about 32 mm, less than about 31.5 mm, less than about 31 mm or less than about 29.5 mm.

As used herein, the term “capsule width” or “capsule's width” refers to the capsule's external diameter.

In one embodiment of the invention, the capsule's width is at least about 9.5 mm. In another embodiment of the invention, the capsule's width is at least about 9.5 mm, at least about 9.6 mm, at least about 9.7 mm, at least about 9.8 mm, at least about 9.8 mm, at least about 10 mm, at least about 10.1 mm, at least about 10.2 mm, at least about 10.3 mm or at least about 10.4 mm.

In one embodiment of the invention, the capsule's width is about 9.5 mm. In another embodiment of the invention, the capsule's width is about 9.5 mm, about 9.6 mm, about 9.7 mm, about 9.8 mm, about 9.8 mm, about 10 mm, about 10.1 mm, about 10.2 mm, about 10.3 mm, about 10.4 or about 10.5 mm.

In one embodiment of the invention, the capsule's width is as least about 9.5 mm and less than about 10.5 mm. In another embodiment of the invention, the capsule's width is as least about 9.5 mm and less than about 10.5 mm, less than about 10.4 mm, less than about 10.3 mm, less than about 10.2 mm, less than about 10.1 mm, less than about 10 mm, less than about 9.9 mm, less than about 9.8 mm, less than about 9.7 mm or less than about 9.6 mm.

In one embodiment of the invention, the capsule's length is at least about 29 mm and the capsule's width is at least about 9.5 mm. In another embodiment of the invention, the capsule's length is at least about 29 mm, at least about 29.5 mm, at least about 30 mm, at least about 30.5 mm, at least about 31 mm or at least about 31.5 mm and the capsule's width is at least about 9.5 mm. In another embodiment of the invention, the capsule's length is at least about 29 mm and at least about 9.5 mm, at least about 9.6 mm, at least about 9.7 mm, at least about 9.8 mm, at least about 9.8 mm, at least about 10 mm, at least about 10.1 mm, at least about 10.2 mm, at least about 10.3 mm or at least about 10.4 mm.

In one embodiment of the invention, the capsule's length is about 29 mm and the capsule's width is at least about 9.5 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 9.5 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 9.6 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 9.7 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 9.8 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 9.9 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 10 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 10.1 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 10.2 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 10.3 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is at least about 10.4 mm.

In one embodiment of the invention, the capsule's length is about 29 mm and the capsule's width is about 9.5 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 9.5 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 9.6 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 9.7 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 9.8 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 9.9 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 10 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 10.1 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 10.2 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 10.3 mm. In another embodiment of the invention, the capsule's length is about 29 mm, about 29.1 mm, about 29.2 mm, about 29.3 mm, about 29.4 mm, about 29.5 mm, about 29.6 mm, about 29.7 mm, about 29.8 mm, about 29.9 mm, about 30 mm, about 30.1 mm, about 30.2 mm, about 30.3 mm, about 30.4 mm, about 30.5 mm, about 30.6 mm, about 30.7 mm, about 30.8 mm, about 30.9 mm, about 31 mm, about 31.1 mm, about 31.2 mm, about 31.3 mm, about 31.4 mm, about 31.5 mm, about 31.6 mm, about 31.7 mm, about 31.8 mm, about 31.9 mm or about 32 mm and the capsule's width is about 10.4 mm.

In one embodiment of the invention, the capsule's total weight, including the outer shell and anti-parkinsonian medication, is no less than about 1750 mg. In one embodiment of the invention, the capsule's total weight, including the outer shell and anti-parkinsonian medication is no less than about 1750 mg, no less than about 1800 mg, no less than about 1850 mg, no less than about 1900 mg, no less than about 1950 mg, no less than about 2000 mg, no less than about 2050 mg, no less than about 2100 mg or no less than about 2150 mg.

In one embodiment of the invention, the capsule's total weight, including the outer shell and anti-parkinsonian medication, is no less than about 1750 mg and no more than about 2200 mg. In one embodiment of the invention, the capsule's total weight, including the outer shell and anti-parkinsonian medication is no less than about 1750 mg and no more than about 2200 mg, no more than about 2150 mg, more than about 2100 mg, more than about 2050 mg, more than about 2000 mg, more than about 1950 mg, more than about 1900 mg, more than about 1850 mg or more than about 1800 mg.

In one embodiment of the invention, the capsule's total weight, including the outer shell and anti-parkinsonian medication, is about 1750 mg. In one embodiment of the invention, the capsule's total weight, including the outer shell and anti-parkinsonian medication is about 1750 mg, no about 1800 mg, no about 1850 mg, no about 1900 mg, no about 1950 mg, about 2000 mg, about 2050 mg, about 2100 mg, about 2150 mg or no about 2200 mg.

In one embodiment of the invention, the capsule's composition comprises an anti-parkinsonian medication. As used herein, an “anti-parkinsonian medication” is a medication useful in the treatment of Parkinson's disease and/or any of the disease's symptoms, whether caused directly by the disease or consequent of side-effects of medication used to treat the condition.

Examples of anti-parkinsonian medication include dopaminergic agents, COMT inhibitors, DOPA decarboxylase inhibitors, MAO-B inhibitors, anticholinergic agents, adenosine antagonists, adamantanes and combinations thereof.

Examples of anti-parkinsonian dopaminergic agents include levodopa, pramipexole, ropinirole, bromocriptine, piribedil, lisuride, apomorphine, pergolide, rotigotine, cabergoline and their salts and esters.

Examples of anti-parkinsonian COMT inhibitors include entacapone, tolcapone, opicapone and their salts and esters.

Examples of anti-parkinsonian DOPA decarboxylase inhibitors include benserazide, carbidopa and their salts and esters.

Examples of anti-parkinsonian MAO-B inhibitors include selegiline, safinamide, rasagiline and their salts.

Examples of anti-parkinsonian anticholinergic agents include orphenadrine, procyclidine, biperiden, trihexyphenidyl, dexetimide, benzetimide, profenamine, metixene, bornaprine, benztropine and their salts and esters.

Examples of anti-parkinsonian adenosine antagonists include istradefylline and its salts.

Examples of anti-parkinsonian adamantanes include amantadine and its salts.

Examples of combinations of anti-parkinsonian medications include combinations of levodopa and carbidopa or benserazide, with, or without, entacapone.

In one embodiment of the invention, the anti-parkinsonian medication is useful to treat a Parkinson's disease sufferer's akinesia. In one embodiment of the invention, the Parkinson's disease sufferer's akinesia has been induced by levodopa. In one embodiment of the invention, the Parkinson's disease sufferer's akinesia occurs during daytime hours. In another embodiment of the invention, the Parkinson's disease sufferer's akinesia is nocturnal akinesia. In another embodiment of the invention, the Parkinson's disease sufferer's akinesia is early morning akinesia (Early Morning OFF, EMO). As used herein, the term “akinesia” refers to the loss or impairment of the power of voluntary movement and includes the forms akinesia, hypokinesia and bradykinesia. Hypokinesia, bradykinesia and akinesia can each be assessed using standard methodologies known in the field, including the Parkinson's Disease Quality of Life Questionnaire, the Unified Parkinson's Disease Rating Scale (UPDRS) Part III (motor examination), the Movement Disorder Society-sponsored revision of the Unified Parkinson's Disease Rating Scale (MDS-UPDRS) Part III (motor examination), the Time-to-ON Questionnaire and by inertia sensors.

In another embodiment of the invention, the anti-parkinsonian medication is useful to treat a Parkinson's disease sufferer's extrapyramidal symptoms. In another embodiment of the invention, the anti-parkinsonian medication is useful to treat a Parkinson's disease sufferer's motor symptoms. In another embodiment of the invention, the anti-parkinsonian medication is useful to treat a Parkinson's disease sufferer's non-motor symptoms. In one embodiment of the invention, the Parkinson's disease sufferer's non-motor symptoms include mood disorders, such as anxiety and depression, impaired thinking and/or cognition, excessive daytime sleepiness (EDS), insomnia, vivid dreams, decreases in sleep quality, akathisia, rapid eye movement sleep behavior disorder (RBD), sleep disordered breathing, periodic limb movements (PLM), circadian rhythm disruption, sleep disorders, dementia, hallucinations, orthostasis, nausea, vomiting, constipation, gastroparesis, drooling, nocturia, urinary urgency, sweating and pain. In one embodiment of the invention, the anti-parkinsonian medication is an adjunct therapy. In one embodiment of the invention, the anti-parkinsonian medication is administered as a daytime therapy, i.e., as a therapy addressing the daytime symptoms of Parkinson's disease. In another embodiment of the invention, the anti-parkinsonian medication is administered as a nighttime therapy, i.e., as a therapy addressing the nighttime symptoms of Parkinson's disease. In another embodiment of the invention, the anti-parkinsonian medication is administered as a continuous therapy, i.e. providing therapy over both daytime and nighttime hours.

In one embodiment of the invention, the treatment of Parkinson's disease includes an improvement in the frequency of episodes of akinesia in PD sufferers and/or in their akinesia questionnaire or scale scores. In one embodiment, the improvement is in the sufferer's UPDRS Part III.1 (speech), the UPDRS Part III.2 (facial expression), the UPDRS Part III.3 (tremor at rest), the UPDRS Part III.4 (action or postural tremor of hands), the UPDRS Part III.5 (rigidity), the UPDRS Part III.6 (finger taps), the UPDRS Part III.7 (hand movements), the UPDRS Part III.8 (rapid, alternating movements of hands), the UPDRS Part III.9 (leg agility), the UPDRS Part III.10 (arising from chair), the UPDRS Part III.11 (posture), the UPDRS Part III.12 (gait), the UPDRS Part III.13 (postural stability) and/or the UPDRS Part III.14 (body bradykinesia and hypokinesia) scores. In one embodiment, the improvement is in the sufferer's MDS-UPDRS Part III.1 (speech), the MDS-UPDRS Part III.2 (facial expression), the MDS-UPDRS Part III.3 (rigidity), the MDS-UPDRS Part III.4 (finger taps), the MDS-UPDRS Part III.5 (hand movements), the MDS-UPDRS Part III.6 (rapid, alternating movements of hands), the MDS-UPDRS Part III.8 (leg agility), the MDS-UPDRS Part III.9 (arising from chair), the MDS-UPDRS Part III.10 (gait), the MDS-UPDRS Part III.12 (postural stability), the MDS-UPDRS Part III.13 (posture), MDS-UPDRS Part III.14 (body bradykinesia and hypokinesia) scores, the MDS-UPDRS Part III.15 (action or postural tremor of hands) and/or the MDS-UPDRS Parts III.17 and 18 (tremor at rest) scores.

In one embodiment of the invention, the treatment of Parkinson's disease includes the treatment of decreases in sleep quality and consists of improvements in PD sufferer's PDSS, in their PDSS-2, in their PSQI, in their SCOPA-S and/or in their NCSDQ scores. In one embodiment of the invention, the treatment consists of an improvement in sufferer's PDSS 1 (overall quality of night's sleep), in their PDSS 2 (sleep onset), in their PDSS 3 (maintenance and insomnia), in their PDSS 4 (nocturnal restlessness of limbs), in their PDSS 5 (fidgeting), in their PDSS 6 (distressing dreams), in their PDSS 7 (hallucinations), in their PDSS 8 (nocturia), in their PDSS 9 (urinary incontinence secondary to akinesia), in their PDSS 10 (numbness or tingling of limbs), in their PDSS 11 (muscle cramps), in their PDSS 12 (morning waking with painful posture), in their PDSS 13 (tremor upon waking), in their PDSS 14 (sleep refreshment), in their PDSS 15 (daytime dozing), in their PDSS-2 1 (overall quality of past week's sleep), in their PDSS-2 2 (sleep onset), in their PDSS-2 3 (maintenance and insomnia), in their PDSS-2 4 (nocturnal restlessness of limbs), in their PDSS-2 5 (fidgeting), in their PDSS-2 6 (distressing dreams), in their PDSS-2 7 (hallucinations), in their PDSS-2 8 (nocturia), in their PDSS-2 9 (discomfort secondary to akinesia), in their PDSS-2 10 (waking secondary to pain in limbs), in their PDSS-2 11 (waking secondary to muscle cramps), in their PDSS-2 12 (morning waking with painful posturing), in their PDSS-2 13 (tremor upon waking), in their PDSS-2 14 (sleep refreshment), in their PDSS-2 15 (waking secondary to snoring or difficulty in breathing), in their PSQI subjective sleep quality, in their PSQI sleep latency, in their PSQI sleep duration, in their PSQI sleep efficiency, in their PSQI sleep disturbance, in their PSQI use of sleep medication, in their PSQI daytime dysfunction, in their SCOPA-S nighttime sleep, in their SCOPA-S daytime sleepiness and/or in their SCOPA-S quality of sleep scores.

In one embodiment of the invention, the capsule comprises at least about 250 mg of an anti-parkinsonian medication. In one embodiment of the invention, the capsule comprises at least about 250 mg, at least about 300 mg, at least about 350 mg, at least about 400 mg, at least about 450 mg, at least about 500 mg, at least about 550 mg, at least about 600 mg, at least about 650 mg, at least about 700 mg or at least about 750 mg of an anti-parkinsonian medication.

In one embodiment of the invention, the capsule comprises at least about 100 mg, at least about 150 mg, at least about 200 mg, at least about 250 mg or at least about 500 mg of levodopa. In one embodiment of the invention, the capsule comprises about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg or about 800 mg of levodopa and salts and esters thereof.

In one embodiment of the invention, the capsule comprises at least about 0.125 mg, at least about 0.25 mg, at least about 0.375 mg, at least about 0.5 mg, at least about 0.75 mg, at least about 1 mg, at least about 1.25 mg, at least about 1.5 mg, at least about 2.25 mg, at least about 3 mg, at least about 3.75 mg or at least about 4 mg of pramipexole. In one embodiment of the invention, the capsule comprises about 0.125 mg, about 0.25 mg, about 0.375 mg, about 0.5 mg, about 0.75 mg, about 1 mg, about 1.25 mg, about 1.5 mg, about 2.25 mg, about 3 mg, about 3.75 mg or about 4 mg of pramipexole.

In one embodiment of the invention, the capsule comprises at least about 0.25 mg, at least about 0.5 mg, at least about 1 mg, at least about 2 mg, at least about 3 mg, at least about 4 mg, at least about 5 mg, at least about 6 mg, at least about 8 mg or at least about 12 mg of ropinirole or salt thereof. In one embodiment of the invention, the capsule comprises about 0.25 mg, about 0.5 mg, about 1 mg, about 2 mg, about 3 mg, about 4 mg, about 5 mg, about 6 mg, about 8 mg or about 12 mg of ropinirole or salt thereof.

In one embodiment of the invention, the capsule comprises at least about 0.8 mg, at least about 2.5 mg or at least about 5 mg of bromocriptine or salt thereof. In one embodiment of the invention, the capsule comprises about 0.8 mg, about 2.5 mg or about 5 mg of bromocriptine or salt thereof.

In one embodiment of the invention, the capsule comprises at least about 50 mg of piribedil. In one embodiment of the invention, the capsule comprises about 50 mg of piribedil.

In one embodiment of the invention, the capsule comprises at least about 0.1 mg or at least about 0.2 mg of lisuride. In one embodiment of the invention, the capsule comprises about 0.1 mg or about 0.2 mg of lisuride.

In one embodiment of the invention, the capsule comprises at least about 10 mg, at least about 15 mg, at least about 20 mg, at least about 25 mg or at least about 30 mg of apomorphine. In one embodiment of the invention, the capsule comprises about 10 mg, about 15 mg, about 20 mg, about 25 mg or about 30 mg of apomorphine.

In one embodiment of the invention, the capsule comprises at least about 0.05 mg, at least 0.25 mg or at least 10 mg of pergolide or salt thereof. In one embodiment of the invention, the capsule comprises about 0.05 mg, about 0.25 mg or about 10 mg of pergolide or salt thereof.

In one embodiment of the invention, the capsule comprises at least about 1 mg, at least about 2 mg, at least about 3 mg, at least about 4 mg, at least about 6 mg or at least about 8 mg of rotigotine. In one embodiment of the invention, the capsule comprises about 1 mg, about 2 mg, about 3 mg, about 4 mg, about 6 mg or about 8 mg of rotigotine.

In one embodiment of the invention, the capsule comprises at least about 0.5 mg of cabergoline. In one embodiment of the invention, the capsule comprises about 0.5 mg of cabergoline.

In one embodiment of the invention, the capsule comprises at least about 10 mg, at least about 25 mg, at least about 37.5 mg or at least about 50 mg of carbidopa. In one embodiment of the invention, the capsule comprises about 10 mg, about 12.5 mg, about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, about 50 mg, about 52.5 mg, about 55 mg, about 57.5 mg, about 60 mg, about 62.5 mg, about 65 mg, about 67.5 mg, about 70 mg, about 72.5 mg, about 75 mg, about 77.5 mg or about 80 mg of carbidopa and salts and esters thereof.

In one embodiment of the invention, the capsule comprises at least about 12.5 mg, at least about 25 mg or at least about 50 mg of benserazide or salt thereof. In one embodiment of the invention, the capsule comprises about 12.5 mg, about 25 mg or about 50 mg of benserazide or salt thereof.

In one embodiment of the invention, the capsule comprises at least about 200 mg of entacapone. In one embodiment of the invention, the capsule comprises about 200 mg of entacapone.

In one embodiment of the invention, the capsule comprises at least about 100 mg of tolcapone. In one embodiment of the invention, the capsule comprises about 100 mg, about 150 mg or about 200 mg of tolacapone.

In one embodiment of the invention, the capsule comprises at least about 50 mg of opicapone. In one embodiment of the invention, the capsule comprises about 50 mg of opicapone.

In one embodiment of the invention, the capsule comprises at least about 1.25 mg or at least about 5 mg of selegiline and salts thereof. In one embodiment of the invention, the capsule comprises about 1.25 mg or about 5 mg of selegiline and salts thereof.

In one embodiment of the invention, the capsule comprises at least about 50 mg or at least about 100 mg of safinamide and salts thereof. In one embodiment of the invention, the capsule comprises about 50 mg or about 100 mg of safinamide and salts thereof.

In one embodiment of the invention, the capsule comprises at least about 0.5 mg or at least about 1 mg of rasagiline and salts thereof. In one embodiment of the invention, the capsule comprises about 0.5 mg or about 1 mg of rasagiline and salts thereof.

In one embodiment of the invention, the capsule comprises at least about 25 mg, at least about 50 mg or at least about 100 mg of orphenadrine or salt thereof. In one embodiment of the invention, the capsule comprises about 25 mg, about 50 mg or about 100 mg of orphenadrine or salt thereof.

In one embodiment of the invention, the capsule comprises at least about 2 mg or at least about 5 mg of procyclidine and salts thereof. In one embodiment of the invention, the capsule comprises about 2 mg or about 5 mg of procyclidine and salts thereof.

In one embodiment of the invention, the capsule comprises at least about 2 mg of biperiden or salt thereof. In one embodiment of the invention, the capsule comprises about 2 mg of biperiden or salt thereof.

In one embodiment of the invention, the capsule comprises at least about 2 mg or at least about 5 mg of trihexyphenidyl and salts thereof. In one embodiment of the invention, the capsule comprises about 2 mg or about 5 mg of trihexyphenidyl and salts thereof.

In one embodiment of the invention, the capsule comprises at least about 0.5 mg or at least about 1 mg of dexetimide. In one embodiment of the invention, the capsule comprises about 0.5 mg or about 1 mg of dexetimide.

In one embodiment of the invention, the capsule comprises at least about 1 mg or at least about 2 mg of benzetimide. In one embodiment of the invention, the capsule comprises about 1 mg or about 2 mg of benzetimide.

In one embodiment of the invention, the capsule comprises at least about 10 mg, at least about 50 mg or at least about 100 mg of profenamine or salt thereof. In one embodiment of the invention, the capsule comprises about 10 mg, about 50 mg or about 100 mg of profenamine or salt thereof.

In one embodiment of the invention, the capsule comprises at least about 2.5 mg of mexitene or salt thereof. In one embodiment of the invention, the capsule comprises about 2.5 mg of mexitene or salt thereof.

In one embodiment of the invention, the capsule comprises at least about 2 mg of bornaprine or salt thereof. In one embodiment of the invention, the capsule comprises about 2 mg of bornaprine or salt thereof.

In one embodiment of the invention, the capsule comprises at least about 0.5 mg, at least about 1 mg or at least about 2 mg of benztropine or salt thereof. In one embodiment of the invention, the capsule comprises about 0.5 mg, about 1 mg or about 2 mg of benztropine or salt thereof.

In one embodiment of the invention, the capsule comprises at least about 20 mg or at least about 40 mg of istradefylline and salts thereof. In one embodiment of the invention, the capsule comprises about 20 mg or about 40 mg of istradefylline and salts thereof.

In one embodiment of the invention, the capsule comprises at least about 50 mg, at least about 68.5 mg, at least about 100 mg, at least about 129 mg, at least about 137 mg, at least about 161 mg, at least about 193 mg or at least about 258 mg of amantadine and salts thereof.

In one embodiment of the invention, the capsule comprises levodopa and a DOPA decarboxylase inhibitor and the (w/w) ratio of levodopa to DOPA decarboxylase inhibitor is 1:4. In another embodiment of the invention, the pharmaceutical composition comprises levodopa and a DOPA decarboxylase inhibitor and the (w/w) ratio of levodopa to DOPA decarboxylase inhibitor is 1:5. In another embodiment, the pharmaceutical composition comprises levodopa and a DOPA decarboxylase inhibitor and the (w/w) ratio of levodopa to DOPA decarboxylase inhibitor is between 1:5 and 1:10. In yet another embodiment, the pharmaceutical composition comprises levodopa and a DOPA decarboxylase inhibitor and the (w/w) ratio of levodopa to DOPA decarboxylase inhibitor is 1:10.

In one embodiment of the invention, the capsule comprises about 10 mg of carbidopa and about 100 mg of levodopa. In another embodiment of the invention, the capsule comprises about 25 mg of carbidopa and about 100 mg of levodopa. In another embodiment of the invention, the capsule comprises about 25 mg of carbidopa and about 250 mg of levodopa. In another embodiment of the invention, the capsule comprises about 50 mg of carbidopa and about 200 mg of levodopa. In another embodiment of the invention, the capsule comprises about 23.75 mg of carbidopa and about 95 mg of levodopa. In another embodiment of the invention, the capsule comprises about 36.25 mg of carbidopa and about 145 mg of levodopa. In another embodiment of the invention, the capsule comprises about 48.75 mg of carbidopa and about 195 mg of levodopa. In another embodiment of the invention, the capsule comprises about 61.25 mg of carbidopa and about 245 mg of levodopa. In another embodiment of the invention, the capsule comprises about 75 mg of carbidopa and about 300 mg of levodopa. In another embodiment of the invention, the capsule comprises about 87.5 mg of carbidopa and about 350 mg of levodopa. In another embodiment of the invention, the capsule comprises about 100 mg of carbidopa and about 400 mg of levodopa. In another embodiment of the invention, the capsule comprises about 112.5 mg of carbidopa and about 450 mg of levodopa. In another embodiment of the invention, the capsule comprises about 125 mg of carbidopa and about 500 mg of levodopa. In another embodiment of the invention, the capsule comprises about 137.5 mg of carbidopa and about 550 mg of levodopa. In another embodiment of the invention, the capsule comprises about 150 mg of carbidopa and about 600 mg of levodopa. In another embodiment of the invention, the capsule comprises about 167.5 mg of carbidopa and about 650 mg of levodopa. In another embodiment of the invention, the capsule comprises about 175 mg of carbidopa and about 700 mg of levodopa. In another embodiment of the invention, the capsule comprises about 187.5 mg of carbidopa and about 750 mg of levodopa. In another embodiment of the invention, the capsule comprises about 200 mg of carbidopa and about 800 mg of levodopa. In another embodiment of the invention, the capsule comprises about 30 mg of carbidopa and about 300 mg of levodopa. In another embodiment of the invention, the capsule comprises about 35 mg of carbidopa and about 350 mg of levodopa. In another embodiment of the invention, the capsule comprises about 40 mg of carbidopa and about 400 mg of levodopa.

In another embodiment of the invention, the capsule comprises about 45 mg of carbidopa and about 450 mg of levodopa. In another embodiment of the invention, the capsule comprises about 50 mg of carbidopa and about 500 mg of levodopa. In another embodiment of the invention, the capsule comprises about 55 mg of carbidopa and about 550 mg of levodopa. In another embodiment of the invention, the capsule comprises about 60 mg of carbidopa and about 600 mg of levodopa. In another embodiment of the invention, the capsule comprises about 65 mg of carbidopa and about 650 mg of levodopa.

In another embodiment of the invention, the capsule comprises about 70 mg of carbidopa and about 700 mg of levodopa. In another embodiment of the invention, the capsule comprises about 75 mg of carbidopa and about 750 mg of levodopa. In another embodiment of the invention, the capsule comprises about 80 mg of carbidopa and about 800 mg of levodopa.

In one embodiment of the invention, the capsule comprises about 12.5 mg of benserazide and about 50 mg of levodopa. In one embodiment of the invention, the capsule comprises about 25 mg of benserazide and about 100 mg of levodopa. In one embodiment of the invention, the capsule comprises about 50 mg of benserazide and about 200 mg of levodopa.

In one embodiment of the invention, the capsule comprises about 25 mg of carbidopa, about 200 mg of entacapone and about 100 mg of levodopa. In another embodiment of the invention, the capsule comprises about 37.5 mg of carbidopa, about 200 mg of entacapone and about 125 mg of levodopa. In another embodiment of the invention, the capsule comprises about 37.5 mg of carbidopa, about 200 mg of entacapone and about 150 mg of levodopa. In another embodiment of the invention, the capsule comprises about 50 mg of carbidopa, about 200 mg of entacapone and about 200 mg of levodopa. In another embodiment of the invention, the capsule comprises about 12.5 mg of carbidopa, about 200 mg of entacapone and about 50 mg of levodopa. In another embodiment of the invention, the capsule comprises about 18.75 mg of carbidopa, about 200 mg of entacapone and about 75 mg of levodopa.

The disclosure also provides, inter alia, methods of treating Parkinson's disease comprising orally administering to a human subject diagnosed with a Hoehn and Yahr rating of at least 2, a capsule comprising at least 400 mg of levodopa and at least 40 mg of carbodopa and wherein the capsule's length is about 29 mm and the capsule's width about 9.5 mm.

In one embodiment of the invention, the method comprises orally administering to a human subject diagnosed with a Hoehn and Yahr rating of at least 2, a capsule comprising about 10 mg of carbidopa and about 100 mg of levodopa, about 25 mg of carbidopa and about 100 mg of levodopa, about 25 mg of carbidopa and about 250 mg of levodopa, about 50 mg of carbidopa and about 200 mg of levodopa, about 23.75 mg of carbidopa and about 95 mg of levodopa, about 36.25 mg of carbidopa and about 145 mg of levodopa, about 48.75 mg of carbidopa and about 195 mg of levodopa, about 61.25 mg of carbidopa and about 245 mg of levodopa, about 75 mg of carbidopa and about 300 mg of levodopa, about 87.5 mg of carbidopa and about 350 mg of levodopa, about 100 mg of carbidopa and about 400 mg of levodopa, about 112.5 mg of carbidopa and about 450 mg of levodopa, about 125 mg of carbidopa and about 500 mg of levodopa, about 137.5 mg of carbidopa and about 550 mg of levodopa, about 150 mg of carbidopa and about 600 mg of levodopa, about 167.5 mg of carbidopa and about 650 mg of levodopa, about 175 mg of carbidopa and about 700 mg of levodopa, about 187.5 mg of carbidopa and about 750 mg of levodopa, about 200 mg of carbidopa and about 800 mg of levodopa, about 30 mg of carbidopa and about 300 mg of levodopa, about 35 mg of carbidopa and about 350 mg of levodopa, about 40 mg of carbidopa and about 400 mg of levodopa, about 45 mg of carbidopa and about 450 mg of levodopa, about 50 mg of carbidopa and about 500 mg of levodopa, about 55 mg of carbidopa and about 550 mg of levodopa, about 60 mg of carbidopa and about 600 mg of levodopa, about 65 mg of carbidopa and about 650 mg of levodopa, about 70 mg of carbidopa and about 700 mg of levodopa, about 75 mg of carbidopa and about 750 mg of levodopa or about 80 mg of carbidopa and about 800 mg of levodopa and wherein the capsule's length is about 29 mm and the capsule's width about 9.5 mm.

In one embodiment of the invention, the capsule is a hard-shell capsule. In another embodiment of the invention, the capsule is a soft-gel capsule.

As used herein, the term “hard-shell capsule” refers to a capsule wherein the outer shell comprises two separate pieces in the form of cylinders, each closed at one end and open at the other and wherein the open end of one of the pieces fits over the open end of the other piece to enclose a solid, liquid or semi-solid inner filling.

As used herein, the term “soft-gel capsule” refers to a capsule wherein the outer shell is a single piece and wherein the outer shell surrounds a liquid or semi-solid inner filling.

As used herein, with regard to the capsule's outer shell, the use of the term “enclosed” or “enclosing” shall be synonymous with similar terms, such as surrounding, contained and containing.

In one embodiment of the invention, the capsule's outer shell comprises gelatin, HPMC, cellulose, pullulan gum, gellan gum and combinations thereof and can include additional excipients such as plasticizers such as glycerol or sorbitol, FD&C colorants and opacifying agents, such as titanium dioxide. Capsule's outer shells typically also comprise a water content of at least 10%.

In one embodiment of the invention, the capsule's composition is contained within the capsule's outer shell. In another embodiment of the invention, the capsule's composition is embedded in the capsule's outer shell matrix. In another embodiment of the invention, the capsule's composition is both contained within and embedded in the capsule's outer shell matrix.

In one embodiment of the invention, the anti-parkinsonian medication is contained within the capsule's outer shell. In another embodiment of the invention, the anti-parkinsonian medication is embedded in the capsule's outer shell matrix. In another embodiment of the invention, the anti-parkinsonian medication is both contained within and embedded in the capsule's outer shell matrix.

In one embodiment of the invention, the capsule's composition comprises granules, pellets, powders, tablets, minitablets, suspensions, pastes, non-aqueous liquids, oils, polyethylene glycols, gastroretentive devices and combinations thereof. Capsule compositions can also comprise a variety of excipients, including diluents, such as lactose, maize starch or calcium sulfate, lubricants and gliants such as magnesium stearate or talc, wetting agents such as sodium lauryl sulfate, disintegrants such as crospovidone, sodium starch glycolate, adsorbents such as magnesium carbonate or magnesium oxide, polyethylene glycols, preservatives such as beta-naphthol, oils such as vegetable oils, soybean oil, mineral oil, cod liver oil, miglyol 812, sorbitan derivatives, ionic surface active agents such as polysorbate 80, dimethyl isosorbide, surfactants, diethylene glycol monoethyl ether, water, alcohol, glycerin, polyvinylpyrrolidone, beeswax, lechitin, gelified oil such as Geloil SC, colorants and opacifiers such as titanium dioxide. Examples of gastroretentive devices include WO2012059815, WO2015187746, WO2015191920, WO2018102799 and WO2021092487, each of which is incorporated, in their entirety, herein by reference.

In one embodiment of the invention, the anti-parkinsonian medication is dispersed within the composition. In one embodiment of the invention, the dispersion is uniform and/or homogeneous throughout the composition. In another embodiment of the invention, the dispersion is neither uniform nor homogeneous throughout the composition.

Swallowing is the act of a series of movements utilized by a subject's body to promote the bolus transportation of materials from their mouth to their stomach. Typically, swallowing can be divided into 3 phases, i) the oral phase, where the bolus is collected in the oral cavity and manipulated into a swallow-ready position followed by activity of the tongue to promote the bolus of material from the oral cavity and into the pharynx, ii) the pharyngeal phase, where the bolus is transported towards the stomach while maintaining protection of the subject's airway and preventing the material from entering the subject's respiratory system, and iii) the esophageal phase, where the bolus is pushed down towards the stomach by muscle contractions.

As used herein, the term “dysphagia” refers to the disturbance of the intake or transport of bolus material, usually food, from the mouth to the stomach. In some circumstances, the bolus material is a medication. Dysphagia can represent a dysfunction at any, or all, of the phases of swallowing, and originates from a wide variety of causes whether mechanical, functional or anatomical. Dysphagia can also be divided into 2 subtypes, oropharyngeal dysphagia, where the oral preparatory, oral and/or pharyngeal phases of swallowing are afflicted and esophageal dysphagia, where the esophageal phase is disturbed. In patients with neurological diseases, oral dysfunction regularly predominates over pharyngeal dysfunction.

Features of dysphagia can include aspiration, which is the entry of saliva, food or gastric secretions into the respiratory system below the level of the vocal folds, drooling, which is the complaint of oral spill from the mouth anteriorly when lip closure is incomplete, leaking, which is the premature loss of the bolus over the tongue base into the pharynx before the swallowing reflex is triggered, delayed triggering of the swallowing reflex, retention, which is the pooling of saliva or other materials, usually food, in the oral cavity, nasal regurgitation, which is the entry of materials into the nose due to incomplete velophayngeal closure or pharyngeal/esophageal stop of the bolus with subsequent overflow into the nasal cavity and laryngeal penetration, where food, saliva or other bolus materials reach the larynx at the levels of the vocal folds. A subject will be considered dysphagic if determined to have one or more of these features.

In Parkinson's disease sufferers, swallowing can be affected in the oral phase by bolus manipulation resulting in accumulation of residue in the oropharyngeal area and only piecemeal swallowing. Impaired tongue movements and chewing often lead to abnormal bolus formation and bradykinesia of the tongue can present in more advanced disease. During the pharyngeal phase, there is wide variability between patients with some showing prolongation of bolus transfer timing and statis in the vallecular space and pyridiform sinuses. In the esophageal phase, impairment is found very frequently and can occur at an early stage of the disease. Such impairment might manifest as gastroesophageal reflux, diffuse esophageal spasms and fragmented peristalsis.

In one embodiment of the invention, the subject has been determined to be dysphagic. In another embodiment of the invention, the subject has been determined to not be dysphagic. In one embodiment of the invention, the subject has been determined to be dysphagic to food and to medications. In another embodiment of the invention, the subject has been determined to be dysphagic to medication but not to food. In another embodiment of the invention, the subject has been determined to not be dysphagic to either food or medication. The determination of whether the subject is, or is not, dysphagic can be made either subjectively and/or objectively by a suitably qualified healthcare professional based on a recent clinical evaluation of the subject and by means known in the art, including dysphagia questionnaires, the Comprehensive Clinical Swallowing Examination (CSE), the Repetitive Saliva Swallowing Test (RSST), the Modified Water Swallowing Test (MWST), food tests, Fiberoptic Endoscopic Evaluation of Swallowing (FEES) and/or VideoFluoroscopic Swallowing Study (VFSS).

Examples of the determination and grading of dysphagia and its possible severity include objective scoring of FESS and/or VFSS findings, such as the FHUR-VF evaluation form and graded ordinary point scales.

In one embodiment of the invention, dysphagia severity can be graded on a scale of 0-3 and between 0 (no signs of dysphagia) and 3 (severe dysphagia with penetration or aspiration of multiple constituencies), with scores 1 representing mild dysphagia, with premature bolus spillage or pharyngeal residue in 2 out of 3 swallows of at least 1 consistency, but without penetration or aspiration and 2 representing moderate dysphagia with penetration or aspiration of 1 consistency. In another embodiment of the invention, dysphagia severity can be graded on a Kyyrim 1994 scale of 0-4, where 0 represents able to eat normal diet/no diet dysphagia, 1 represents being able to swallow semi solid foods, 2 represents being able to swallow only semisolid foods, 3 represents being able to swallow liquids only and 4 represents being unable to swallow anything/total dysphagia. In another embodiment of the invention, dysphagia severity can be graded on an Earlam and Cunha-Melo 1981 scale of 1-5, where 1 represents the ability to eat everything but with difficulty, 2 represents dysphagia to solids, 3, dysphagia to semi-solids, 4, dysphagia to fluids and a score of 5 represents absolute dysphagia. In another embodiment of the invention, dysphagia severity can be graded on a Takita 1977 scale of 1-6 where a score of 1 represents complaints of dysphagia but still eating normally, 2 where the subject requires liquids with meals, 3 where the subject is able to swallow semisolids but not solids, 4 where the subject can only swallow liquids, 5 where the subject is unable to swallow liquids but able to swallow saliva and a score of 6 where the subject is unable to swallow saliva. In yet another embodiment of the invention, dysphagia severity is graded according to the CTCAE system rankings.

In one embodiment of the invention, changes in dysphagia, whether relating to changes in efficacy or safety, can be graded using an ordinary point scale. For example, for a graded efficacy score, 0 would represent no impairment with the medication swallowed completely during the first swallowing attempt and the highest score would represent very severe impairment with the medication not swallowed completely and not perceived by the patient, and a graded safety score, 0 would represent no impairment with the medication easily swallowed without any risk of penetration or aspiration and the highest score would represent very severe impairment with the medication or water penetrating into the laryngeal vestibule without attempts to clear it or it being aspirated (Kennedy 1993, Hannig 1995, O'Neil 1999, Buhmann 2019).

In one embodiment of the invention, the subject complains of difficulty swallowing. In one embodiment of the invention, the subject has recently complained to a suitably qualified healthcare professional of difficulty swallowing. In one embodiment of the invention, a suitably qualified healthcare professional has determined that the subject has difficulty swallowing.

The disclosure also provides, inter alia, methods of improving the swallowability of an anti-parkinsonian medication in a human subject diagnosed with a Hoehn and Yahr rating of at least 2, comprising administering the anti-parkinsonian medication to the subject within a capsule and wherein the capsule's length is at least about 29 mm and the capsule's width at least about 9.5 mm.

Examples of the improvement in swallowability of an anti-parkinsonian medication include improvements in the subject's subjective and/or objective swallowability and/or dysphagia efficacy and/or safety scores in comparison to the subject's scores either at baseline or in comparison after swallowing a composition comprising an anti-parkinsonian medication wherein the composition is an oral solid dosage form other than a capsule of length at least about 29 mm and of width at least about 9.5 mm.

In one embodiment of the invention, the subject has previously been determined to have difficulty swallowing smaller dosage forms than the capsule. In one embodiment of the invention, the improvement is in a subject who complains of difficulty swallowing. In one embodiment of the invention, the improvement is in a subject who has recently complained to a suitably qualified healthcare professional of difficulty swallowing. In one embodiment of the invention, improvement is in a subject that a suitably qualified healthcare professional has determined has difficulty swallowing.

The present disclosure will be better understood by reference to the Examples, which follow, but those skilled in the art will readily appreciate that the specific experiments detailed are only illustrative as described more fully in the claims which follow thereafter.

### Example 1: Open-Labelled, Randomized, Study to Evaluate the Swallowability of Elongated Capsules in Patients with Parkinson's Disease

Fifteen patients (11 male, 4 female) with an average age of 70 and each diagnosed with Parkinson's disease and a Hoehn and Yahr score of at least 2, underwent a series of evaluations to assess their ability to swallow small and large capsules and tablets. Following a screening questionnaire addressing their possible dysphagia, each subject underwent a flexible endoscopic evaluation of swallowing (FEES) assessment consisting of 3 attempts to swallow solids, semi-solids and liquids. This assessment provided a baseline reading of the patient's dysphagia. Following the baseline assessment, each patient underwent a second FESS assessment consisting of 2 attempts to swallow each of i) a long gelatin capsule (29 length, 9.6 width, Lonza), ii) a short gelatin capsule (19.4 length, 6.8 width, local pharmacy preparation), iii) a round tablet (10 mm diameter, Winthrop) and iv) an elongated oval tablet (17 mm length, 8.2 mm width, Fagron). All dosage forms were filled or formulated with inactive excipients with the design of both tablets approximated to commercially available anti-parkinsonian medications (Madopor-T® and Stalevo®).

The medication swallowability FESS assessment was graded on 2, 5-point ordinary scales, for efficacy and safety. For efficacy, 0 represented no impairment with the medication swallowed completely during the first swallowing attempt and 4 represented very severe impairment with the medication not swallowed completely and not perceived by the patient. For safety, 0 represented no impairment with the medication easily swallowed without any risk of penetration or aspiration and 4 represented very severe impairment with the medication or water penetrating into the laryngeal vestibule without attempts to clear it or it being aspirated.

Following the second FESS swallowability assessment, each subject completed a second questionnaire addressing how much difficulty did they notice when swallowing the medication? On a scale from 0 (no difficulty at all) to 10 (extreme difficulty) and how anxious did they feel when swallowing the medication? On a scale from 0 (not anxious at all) to 10 (extremely anxious).

Of the fifteen patients, the initial FESS assessment revealed mild dysphagia in 9 subjects (60%) and moderate dysphagia in 1 subject (6.7%). The remaining subjects did not present objectively with dysphagia. The results of the FESS efficacy assessment are shown in Table 1 and demonstrate that the long capsule had better swallowing efficacy than any of the other dosage forms tested.

TABLE 1

| | Long capsule | Short capsule | Round tablet | Oval tablet |
|---|---|---|---|---|
| no impairment | 14 | 11 | 8 | 10 |
| mild impairment | — | 1 | 6 | 3 |
| moderate impairment | 1 | 3 | — | — |
| severe impairment | — | — | — | 1 |
| very severe | — | — | 1 | 1 |
| impairment | | | | |

The results of the FESS safety assessment are shown in Table 2 and demonstrate that the long capsule had better swallowing safety than any of the other dosage forms tested.

TABLE 2

| | Long capsule | Short capsule | Round tablet | Oval tablet |
|---|---|---|---|---|
| no impairment | 14 | 11 | 10 | 11 |
| mild impairment | 1 | 3 | 5 | 3 |
| moderate impairment | — | 1 | — | 1 |
| severe impairment | — | — | — | — |
| very severe | — | — | — | — |
| impairment | | | | |

The results of the safety questionnaire are shown in Table 3 (average score) and demonstrate that the long capsule had similar subjective difficulty and anxiety to any of the other dosage forms tested.

TABLE 3

| | Long capsule | Short capsule | Round tablet | Oval tablet |
|---|---|---|---|---|
| subjective difficulty | 1.5 | 1.6 | 1.8 | 2.4 |
| subjective anxiety | 1.1 | 0.6 | 0.1 | 0.6 |

### Example 2: Clinical Study of Anti-Parkinsonian Medication

The efficacy and safety of anti-parkinsonian capsule medication comprising 500 mg levodopa and 125 mg carbidopa and wherein the capsule's length is about 29 mm and the capsule's width about 9.5 mm, is assessed in a 12 week placebo controlled, double blind study of 240 patients. At baseline, patients have a Hoehn and Yahr rating of at least 2, at least 2 hours per day of OFF time and experience morning akinesia at least 15 days of every month. Prior to initiation of the study, all patients complete a dysphagia questionnaire and undergo a flexible endoscopic evaluation of swallowing (FEES) assessment to determine their baseline subjective and objective levels of dysphagia. Patients are further randomized to receive their medication, either placebo capsule (120 patients) or the anti-parkinsonian capsule medication (120 patients), either within an hour of taking their evening meal or in the hour preceding their going to sleep and all patients undergo a flexible endoscopic evaluation of swallowing (FEES) assessment on at least one occasion during the study period to assess the capsule's swallowability. Upon completion of the study, all patients complete a second dysphagia questionnaire addressing their subjective difficulty and anxiety in swallowing the capsules. The primary endpoint is a change from placebo in the Unified Parkinson's Rating Scale (UPDRS) Part III motor score at 30 minutes after waking the following morning. Secondary endpoints include the comparability of subjective and objective swallowing and safety between both the baseline dysphagic and non-dysphagic populations.

1. A method of administering an anti-parkinsonian medication to a human subject diagnosed with a Hoehn and Yahr rating of at least 2, comprising orally administering to the human subject a capsule comprising an outer shell and a composition comprising the anti-parkinsonian medication; wherein the capsule's length is at least about 29 mm and the capsule's width at least about 9.5 mm.

2. The method of claim 1, wherein the capsule is a hard-shell capsule

3. The method of claim 1, wherein the capsule is a soft-gel capsule.

4. The method of claim 1, wherein the outer shell comprises gelatin, HPMC, cellulose, pullulan gum, gellan gum and combinations thereof.

5. The method of claim 1, wherein the anti-parkinsonian medication is selected from the group consisting of a dopaminergic agent, a COMT inhibitor, a DOPA decarboxylase, a MAO-B inhibitor, an anticholinergic agent, an adenosine antagonist, an adamantane and combinations thereof.

6. The method of claim 5, wherein the dopaminergic agent comprises levodopa.

7. The method of claim 1, where the capsule comprises at least 250 mg of the anti-parkinsonian medication.

8. The method of claim 1, wherein the capsule's length is less than about 32 mm.

9. The method of claim 1, wherein the capsule's width is less than about 10.5 mm.

10. The method of claim 1, wherein the composition comprising the anti-parkinsonian medication is contained within the capsule's outer shell.

11. The method of claim 1, wherein the composition is in the form of a granule, a pellet, a powder, a tablet, a minitablet, a suspension, a paste, a non-aqueous liquid, an oil, a polyethylene glycol, a gastroretentive device and combinations thereof.

12. The method of claim 1, wherein the anti-parkinsonian medication is dispersed within the composition.

13. The method of claim 12, wherein the dispersion is uniform and/or homogeneous.

14. The method of claim 1, wherein the subject has been further determined as dysphagic.

15. The method of claim 1, wherein the subject has been further determined to not be dysphagic.

16. The method of claim 1, wherein the subject has difficulty swallowing.

17. The method of claim 1, for the treatment of the subject's akinesia.

18. The method of claim 1, for the treatment of the subject's extrapyramidal symptoms.

19. The method of claim 1, for the treatment of the subject's motor symptoms.

20. The method of claim 1, for the treatment of the subject's non-motor symptoms.

21. The method of claim 1, wherein the administration is a daytime therapy.

22. The method of claim 1, wherein administration is a nighttime therapy.

23. The method of claim 1, wherein the administration is a continuous therapy.

24. A method of treating Parkinson's Disease in a human subject diagnosed with a Hoehn and Yahr rating of at least 2, comprising orally administering to the subject a capsule comprising at least 400 mg of levodopa and at least 40 mg of carbodopa and wherein the capsule's length is about 29 mm and the capsule's width about 9.5 mm.

25. A method of treating Parkinson's Disease in a human subject comprising the method of claim 1.

26. A method of improving the swallowability of an antiparkinsonian medication in a human subject diagnosed with a Hoehn and Yahr rating of at least 2, comprising administering the antiparkinsonian medication to the subject within a capsule and wherein the capsule's length is at least about 29 mm and the capsule's width at least about 9.5 mm.

27. The method of claim 26, wherein the subject has previously been determined to have difficulty swallowing dosage forms smaller than the capsule.
